(19) United States
(12) Patent Application Publication
(10) Pub. No.: US 2007/0213425 A1
(43) Pub. Date: Sep. 13, 2007

(54) Modified bone cement

(75) Inventors: Paul Higham, Ringwood, NJ (US); Shulin He, Waldwick, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 11/370,681

(22) Filed: Mar. 8, 2006

(51) Int. Cl. A61K 6/08

(52) U.S. Cl. 523/116

(57) ABSTRACT

An acrylic bone cement composition, including a liquid monomer component and a polymer powder component in the form of beads. The liquid monomer component includes a methylmethacrylate polymer and the polymer component includes a polymethylmethacrylate polymer powder, wherein the polymer powder component is subjected to a milling process.

### BACKGROUND OF THE INVENTION

The present invention relates to an improved surgical bone cement composition and more particularly to a method of forming the bone cement.

Various types of bone cements have been used for securing prosthesis. Generally speaking, known bone cements consist of a polymer component (bead-shaped acrylate or methacrylate pre-polymers termed “bead-polymers” or “pearl polymers”), a monomeric acrylic or methacrylic acid derivative, e.g., methyl methacrylate, a polymerization catalyst and possibly a stabilizing and activating agent or accelerator. Prior to use, the components are mixed to a homogenous mass and brought to the site of application in a suitable way, such as by a cement injection device. The monomeric component hardens by polymerization and thereby encloses the pre-polymer. A homogenous polymer structure results. Polyacrylic-bone cements are not absorbable. A stable connection between the implant and the implant bed is given by the interlocking feature of the bone cement and tissue ingrowth.

Once the various components of the bone cement are mixed together, the bone cement has a liquid or doughy consistency and can be applied during this working period. As time lapses, the bone cement begins to cure and harden. Therefore, it is only during the time that the bone cement has a doughy consistency that it may be applied. However, as more efficient ways for introducing the bone cement into cavities and the like develop, the required time frame of the bone cement being a dough-like consistency is reduced. And by reducing the dough-time, the setting-time (time for the cement to completely cure) is reduced as well as the time for the entire surgical operation. A reduced operation time provides many advantages including limiting the susceptibility of the patient to infection and other surgical complications.

### SUMMARY OF THE INVENTION

The present invention is directed to an acrylic bone cement. In one specific embodiment, the bone cement has a composition including a liquid monomer component and a polymer powder component in the form of beads having an exterior surface. The liquid monomer component includes a methyl methacrylate monomer and the polymer component includes a poly methyl methacrylate polymer powder. The polymer powder component beads are subsequently milled to roughen up the exterior surface of the beads. When the liquid monomer component is mixed with a polymer powder component to form a resulting cement, the resulting cement has a dough-time that is less than the dough-time of the cement that includes the same mixture, except that the polymer powder component was not milled.

The acrylic bone cement and specifically all of the polymer powder beads or at least substantially all, may be milled to roughen up the surface of the polymer powder beads. The beads may be milled using a ball mill, a jet mill or the like.

In addition to the materials previously mentioned, the acrylic bone cement may also include barium sulfate.

In one aspect of the present invention, prior to milling, the polymer powder beads have a first surface area measured in square meters per gram and after milling, the polymer beads have a second surface area measured in square meters per gram. The second surface area being at least twice as large as the first surface area.

The liquid monomer component to polymer powder component ratio is preferably one to two, volume per weight. The polymer beads may have substantially the same shape after milling as compared to before milling. The resultant cement preferably has a tensile strength that is substantially equal to the tensile strength of a bone cement comprised of the same mixture of the resultant cement, except that the mixture does not include milling the polymer powder component. The polymer powder beads are exposed to a sufficient amount of force to roughen the exterior surface of the beads, but are not exposed to a sufficient amount of force to crush them.

### BRIEF DESCRIPTION OF THE DRAWINGS

FIG. 1A is a field emission scanning electronic microscopy photograph of a polymer powder bead prior to the bead being subjected to a grinding process;

FIG. 1B is similar photograph after the bead has been subjected to a grinding process;

FIG. 2A is an atomic force microscopy illustration of a surface of a polymer powder bead prior to being subjected to a grinding process; and

FIG. 2B is an atomic force microscopy illustration of the surface of the bead of FIG. 2A after the bead has been subjected to a grinding process.

### DETAILED DESCRIPTION

The present invention relates to a self-curing bone cement formed as a polymeric reaction product after mixing a powdered polymer component with a liquid monomer component. More particularly, the present invention relates to a method for preparing the bone cement. Bone cements are generally formed using acrylic cement-like substances. For example, a liquid monomer in one particular embodiment may be a methyl methacrylatic monomer, and a polymer powder may be a poly methyl methacrylate (“PMMA”) or a PMMA copolymer powder. An example of such a bone cement is Surgical Simplex® P sold by Stryker Howmedica Osteonics. Surgical Simplex® P is typically comprised of a liquid component to powder component ratio of one to two (volume/weight) where the volume is in milliliters and the weight is measured in grams. When mixing the liquid and powder components, the liquid is added to the powder to form a dough-like mass. In a preferred embodiment the polymer is in the shape of beads and preferably is styrene copolymer powder beads. The styrene copolymer beads are preferably 75% w/w.

In one aspect of the present invention, prior to the mixing of the copolymer beads with the liquid monomer, the beads subjected to a milling process. In one such embodiment, the beads are placed in a ball mill to grind and roughen the surfaces of the beads. A ball mill actuates the beads by tumbling the beads against a grinding medium such as balls, rods or the like. The ball mill used in the present invention comprised a ceramic jar filled with ceramic balls up to 75% in volume. The ball mill jar was made by US Stoneware.

During various experiments, a first trial was conducted where 75 ml polymer beads were placed in a 300 ml ceramic jar packed with 150 ml ceramic bars. The polymer beads are drilled milled on the roller at approximately 300 rpm. The jar is then rolled on a roller such that the copolymer beads placed within the jar are subjected to a grinding process. Three different samples of copolymer beads were ground for one hour, two hours and four hours respectively, i.e., Batch A, Batch B and Batch C. Each collected batch was then separately mixed with additional ingredients to begin to formulate the cement. For instance, 30 g of the grounded copolymer beads of Batch A subjected to a 1 hour grinding process were blended with 6 g of PMMA and 4 g of barium sulfate on a roller blender. The prepared cement was sterilized by irradiation at a production dose (30-40 kGy). This formulation of the cement is equivalent to the formation used to produce the Surgical Simplex® P bone cement. Surgical Simplex® P bone cement consists of powder and liquid components. A single dose of cement has 40 grams of powder and 20 ml of liquid. The powder component consists of 75% w/w of methyl methacrylate-styrene copolymer (beads)(containing benzoyl peroxide 1.7% w/w); 15% w/w of polymethyl methacrylate; and 10% w/w barium sulfate. The benzoyl peroxide acts as an initiator. The liquid component is comprised of: 97.4% v/v of methyl methacrylate; 2.6% v/v N,N-dimethyl-p-toluidine; and 75±15 ppm of hydroquinone. Other bone cements include Palacos R bone cement, Smartset HV, Endurance Bone cement

After Batch A with beads milled for 1 hour was prepared, the same formulation was also used to prepare additional bone cement mixtures using the polymer beads of Batch B and Batch C. Batch B and Batch C were subjected to the grinding process for 2 hours and 4 hours, respectively. To reiterate, the batch subjected to a ball mill process for 1 hour is labeled Batch A; for 2 hours, Batch B; and for 4 hours, Batch C. The three batches, A, B and C, subjected to the ball milling process for different time periods, as well as a batch comprised of non-ground beads, Batch D, were then tested for their setting-time, dough-time and maximum temperature according to the standards of the ASTM F451-95/ISO 5833:1992 method. In order to measure the dough-time Encore orthopaedic surgical gloves were used to mix the components. All experiments were conducted in an environmental control room at 18.50° C. (65° C.) and 50% relative humidity.

The respective properties of the four batches, A, B, C, and D are shown in Table 1. The results illustrate that both the dough-time and setting-time are significantly affected by grinding the copolymer. The dough-time and setting-time for Batch D is 5 minutes and 18.4 minutes, respectively. As can be seen by a review of Table 1, by increasing the grinding time of the copolymer beads, the dough-time and setting-time are decreased. This is highlighted when comparing the specific batches that were milled. For instance, Batch A, which was ball milled for 1 hour has a dough-time of 4.25 minutes and a setting-time of 16.2 minutes. Both time measurements are less than the time measurements for the unmilled batch, Batch D. And when the milling time was increased to two hours, i.e. Batch B, the dough-time dropped to 3.5 minutes and the setting-time dropped to 15.1 minutes. Further, with the grinding time increased to 4 hours, i.e. Batch C, the dough-time and setting-time are decreased to 3.25 minutes and 13.7 minutes respectively. Since the increase in grinding time is directly proportional to an increase in surface roughness, i.e., surface area of the beads, the results further illustrate that an increase in surface area of the beads decreases the dough-time and setting-time of the cement. The decrease in dough-time and setting-time as for example when comparing Batch D, which was not subjected to a mill process and Batch C, which was milled for 4 hours may be attributed to the increase in surface roughness of the polymer beads. This was verified via testing, where it was ascertained that polymer beads that were subjected to 4 hours of a ball mill process had a average surface area or 0.2203 m²/g as compared to the polymer beads that were not milled and had an average surface area or 0.1848 m²/g.

TABLE 1
Ball Milled Batches

| | Grinding | Dough-Time | Setting- | |
|---|---|---|---|---|
| Batch | Time (hour) | (min) | Time (min) | Tmax (° C.) |
| A | 1 | 4.25 | 16.2 | 68.7 |
| B | 2 | 3.50 | 15.1 | 72.4 |
| C | 4 | 3.25 | 13.7 | 65.9 |
| D | 0 | 5.00 | 18.4 | 62.3 |

In an alternate method of operation, copolymer beads are ground in a jet mill as compared to a ball mill. Jet mills operate by creating a high speed stream of air that circulates about a torodial chamber. The feed product, such as beads, may be introduced into the air stream. As the feed product is continually rotated around the chamber, individual particles of the feed product contact other individual particles of the product. In the case of copolymer beads, as the particles collide and their surfaces contact one another, the surfaces become pitted or roughened. A jet mill is considered cleaner and more efficient than alternate milling processes and therefore limits the risk of potential contamination of the product being milled. The jet mill employed in conjunction with the present invention is Model: Netzsch-Condux Fluidized Bed Jet Mill CGS 16, made by NETZSCH Incorporated. Similar to the experiments carried out using the ball milling process, four separate batches of copolymer beads were subjected to the jet milling process for different periods of time, or at different speeds, or a combination of the both. For instance, the beads used in Trial 1 were ground for 15 minutes in the jet mill at a classifier speed of 2030 RPM and the beads of Trial 2 were ground for 10 minutes at a classifier speed of 1530 RPM. The parameters of the grinding conditions employed for all of the Trials, Trial 1, 2, 3, and 4, subjected to the jet milling process are illustrated in Table 2 reproduced below.

TABLE 2
Jet Mill Parameters

| | |
|---|---|
| Grinding | Grinding Trial |

| parameter | Trial 1 | Trial 2 | Trial 3 | Trial 4 |
|---|---|---|---|---|
| Time (min) | 15 | 10 | 10 | 10 |
| Classifier | 2030 | 1530 | 1200 | 900 |
| speed (RPM) | | | | |
| Classifier | 12.7 | 9.6 | 9.6 | 9.6 |
| tip speed | | | | |
| (m/s) | | | | |
| Power: load | 0.08 | 0.08 | 0.08 | 0.08 |
| (kw) | | | | |
| Blower speed | 2235 | 2355 | 2355 | 2355 |
| actual RPM | | | | |
| Grinding air | 90 | 94 | 94 | 94 |
| pressure | | | | |
| (PSI) | | | | |
| Grinding | 12 | 14 | 14 | 14 |
| temperature | | | | |
| (C.) | | | | |
| Airflow | 118.7 | 122.8 | 122.8 | 122.8 |
| (m3/hr) | | | | |
| Bed weight | 11.4 | 11.4 | 11.4 | 11.4 |
| actual (kg) | | | | |

The particle size of the polymer beads for each of the Trials illustrated in Table 2 are shown in Table 3.

TABLE 3
Particle size for Beads Subjected to a Jet Mill

| Particle size | | Control | Trial 1 | Trial 2 | Trial 3 | Trial 4 |
|---|---|---|---|---|---|---|
| Particle | Mean | 35.09 | 16.38 | 32.98 | 39.21 | 51.06 |
| size | D10 | 18.21 | 9.24 | 20.27 | 19.30 | 29.72 |
| (micron) | D25 | 27.39 | 13.48 | 26.97 | 30.12 | 42.40 |
| | D50 | 38.30 | 18.70 | 34.51 | 43.62 | 56.94 |
| | D75 | 49.47 | 24.34 | 42.91 | 57.40 | 68.91 |
| | D90 | 58.64 | 29.52 | 51.52 | 68.25 | 77.72 |

Table 3 is a summary of the particle size distribution of the Control polymer beads as well as the particle size distribution of each trial subjected to the jet mill process at differing parameters. The particle size distribution was measured using a Beckman Coulter LS32 130 equipped with a Tornado system. The mean particle size is the average particle size in volume. For instance, D10 means 10% of the particles in volume. The D10 for the control batch is 18.21 microns, which translates to 10% of the particles in volume of the control batch have a volume that is less than 18.21 microns. Trials 1, 2, 3, and 4 of Table 3 were generated by grinding the same control batch with different grinding parameters listed in Table 2.

Similar to the Batches A, B and C having the copolymer beads subjected to a ball milling process, the four Trials of copolymer beads 1, 2, 3 and 4 subjected to the jet milling process are then mixed with additional ingredients, the ingredients being equivalent to those used in the formation of Surgical Simplex® P bone cement. The resultant cements are then analyzed with specific interest paid to the dough-times, setting-times and maximum temperatures of the individual Trials. The resultant characteristics are summarized in Table 4 reproduced below.

TABLE 4

| Grinding trial | Dough-time (min) | Setting-time (min) | Tmax(C. °) |
|---|---|---|---|
| Control | 6.00 | 19.00 | 60 |
| Trial 1 | 2.75 | 10.15 | 50 |
| Trial 2 | 3.67 | 10.86 | 73 |
| Trial 3 | 4.00 | 13.7 | 62 |
| Trial 4 | 5.17 | 16.5 | 52 |

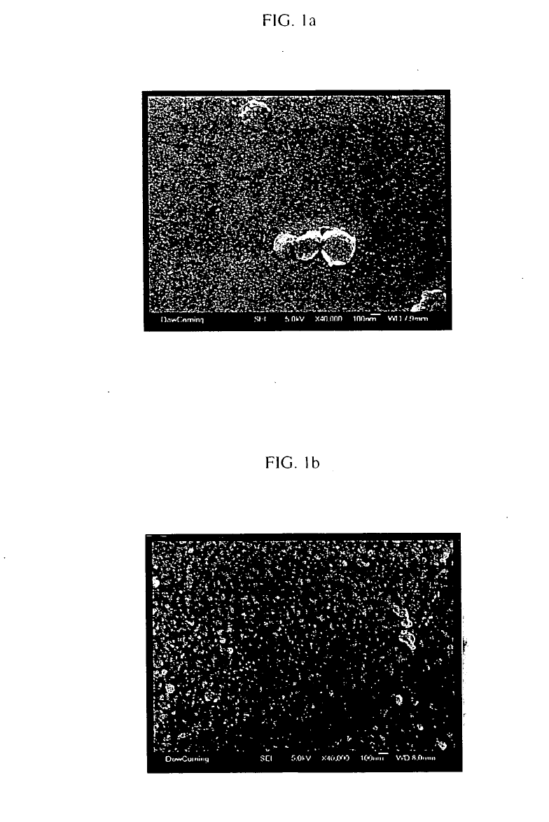

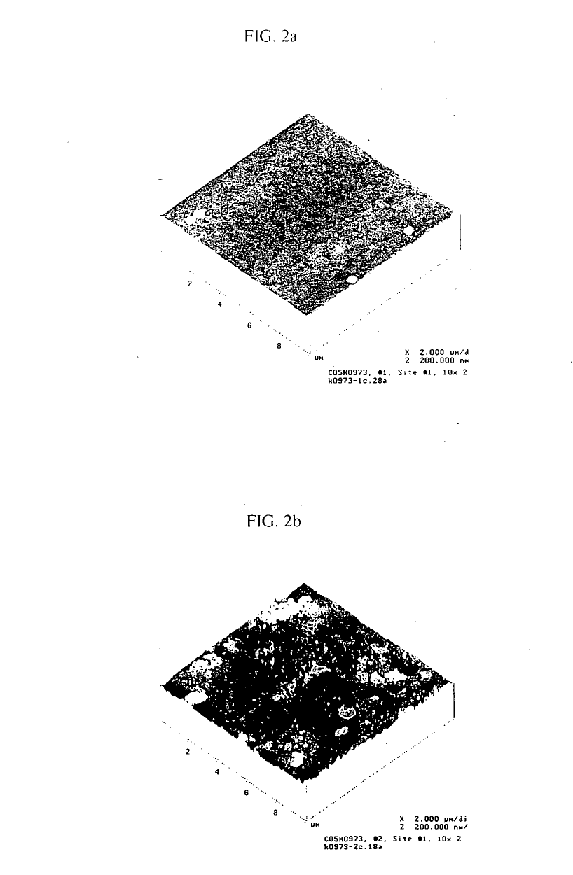

Prior to preparing the mixtures, the Trials of ground polymers were subjected to analysis. SEM images show that the jet milling process does not change the shape of the spherical beads, but field emissions scanning electric microscopy (FESEM) illustrate that grinding significantly increased the surface roughness of the polymer beads as shown in FIGS. 1A and 1B. FIG. 1A is a photographic image of the surface of a bead that was not subjected to a jet grinding process, while FIG. 1B is a photographic image of the surface of a bead after being ground in the jet mill. The beads in FIG. 1B are from Trial 2 of Table 2. In addition, atomic force microscopy (AFM) as shown in FIGS. 2A and 2B further confirms that the mean surface roughness of the ground beads is about 4 times higher than that of beads that are not subjected to a grinding process. Similar to FIGS. 1A and 1B, FIG. 2A illustrates the surface of a bead not ground and FIG. 2B illustrates the surface of a bead subjected to a grinding process. As with FIG. 1B, the beads in FIG. 2B are taken from Trial 2 that was subjected to the jet mill process. Table 5 illustrates the BET surface area for each of the copolymer beads that were in individual Trials and subjected to the jet mill process and the surface area of control copolymer beads that were not ground.

TABLE 5
BET surface area (m²/g)

| | | BET surface | |
|---|---|---|---|
| | | area (m²/g) | Increase (%) |
| Before grinding (control) | | 0.1848 | 0 |
| Grinding Trial | 1 | 0.4507 | 144 |
| | 2 | 0.5040 | 173 |
| | 3 | 0.3362 | 82 |
| | 4 | 0.2103 | 14 |

By analyzing Table 5 in conjunction with Table 3 and Table 4, it can be determined that the dough-time and setting-time of the bone cement mixture is directly related to the surface area of the copolymer beads used in the bone cement. For instance, Trials 1 and 2 which have the largest BET surface area at 0.4507 m²/g and 0.5040 m²/g₁ respectively for the beads have the shortest dough-time at 2.75 minutes and 3.67 minutes respectively and shortest setting-time a 10.15 minutes and 10.86 minutes respectively. In contrast, Trials 3 and 4 which have beads with much smaller BET surface area 0.3362 m²/g and 0.2103 m²/g₁ respectively have longer dough-times and setting-times.

The dough-time of the acrylic bone cement depends on the physical process of the mixing between the methyl methacrylate and the powder. A fast dough time indicates quick absorption of the methyl methacrylate by the powder. For this reason, small particle sizes of copolymer usually give a faster dough time than large sizes due to their high surface area. However, the particle size data of the ground materials show the fast dough-time is not necessarily attributed to the particle size of the ground material as Table 3 illustrates. Trials 3 and 4 have larger particle sizes than the control, but-their dough-times and setting-times are much shorter.

Besides setting-time and dough-time, other mechanical properties that are extremely relevant for acrylic bone cement, include the tensile stress of the bone cement. Thus, various tests were conducted on the bone cement formed using the copolymer beads subjected to a grinding process in order to ascertain any affects of the grinding process. The result in tensile stresses of the ground cement and a control specimen are shown in Table 6, illustrated below.

TABLE 6

| Cement | Stress (MPa) | Modulus (MPa) |
|---|---|---|
| Control | 38.33 ± 3.69 | 3149 ± 409 |
| Ground (trial 2)* | 41.62 ± 6.34 | 3050 ± 47 |

*Table 2

Analysis of Table 6 illustrates that the tensile strength and modulus between the control subject and the cement formed using the grounded copolymers have no statistically significant difference. Thus, the grinding process under the conditions of this study does not affect the tensile properties of the cured cement.

In summary, this study demonstrates that a grinding technology that does not break the polymer bead, but instead increases the surface roughness and the surface areas of the polymer beads can be used to decrease the dough-time and setting-time of a bone cement without sacrificing any strength characteristics. Although it is possible to grind the polymer beads to break them, a breaking of such is not required and preferably does not take place during the grinding process.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An acrylic bone cement composition comprising a liquid monomer component and a polymer powder component in the form of beads having a surface, wherein said liquid monomer component includes a methyl methacrylate monomer and said polymer powder component includes a poly methyl methacrylate polymer powder, wherein at least 50% of said polymer powder component beads are subsequently milled to roughen up said surface of said beads, and whereby when said liquid monomer component is mixed with said polymer powder component to form a resultant cement, said resultant cement having a dough-time that is less than the dough-time of a cement that includes the same mixture except that said polymer powder component was not milled.

2. The acrylic bone cement of claim 1, wherein substantially all of said polymer powder beads are milled to roughen up said surface of said polymer powder beads.

3. The acrylic bone cement of claim 1, wherein said polymer powder beads are milled in a ball mill.

4. The acrylic bone cement of claim 1, wherein said polymer powder beads are milled in a jet mill.

5. The acrylic bone cement of claim 1, further comprising barium sulfate.

6. The acrylic bone cement of claim 1, wherein prior to milling, said polymer powder beads have a first surface area measured in square meters per gram, wherein after milling said polymer powder beads have a second surface area measured in square meters per gram, said second surface area being at least twice as large as said second surface area.

7. The acrylic bone cement of claim 1, wherein said liquid monomer component to polymer powder component ratio is 1 to 2 (volume weight).

8. The acrylic bone cement of claim 1, wherein said polymer powder beads have substantially the same shape after milling as compared to before milling.

9. The acrylic bone cement of claim 1, wherein said resultant cement has a tensile strength that is substantially equal to a tensile strength of a bone cement comprised of the same mixture of said resultant cement except that said mixture does not include milling said polymer powder component.

10. A method of preparing an acrylic bone cement comprising the steps of:
milling-a polymer powder-bead component such that an exterior surface of each bead is roughened up;
providing a liquid monomer component to said milled polymer powder bead components such that the resultant cement has a dough-time that is less than a dough-time of a cement formed from the same mixture except that the polymer powder beads where not milled.

11. The method of claim 10, wherein during said milling step said polymer powder beads are exposed to a sufficient amount of force to roughen the exterior surfaces of the beads but are not exposed to a sufficient amount of force to crush them.
